(19) United States
(12) Patent Application Publication
(10) Pub. No.: US 2005/0010065 A1
(43) Pub. Date: Jan. 13, 2005

(54) Method for producing (meth) acrylic acid esters of polyhydric alcohols

(76) Inventors: Alexander Wartini, Heidelberg (DE); Matthias Dernbach, Dossenheim (DE); Jurgen Schroder, Ludwigshafen (DE); Friedrich-Georg Martin, Heidelberg (DE); Tilman Sirch, Schifferstadt (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/494,522

(22) PCT Filed: Nov. 8, 2002
(86) PCT No.: PCT/EP02/12491

(30) Foreign Application Priority Data
Nov. 15, 2001 (DE) 101 56 116.4

(51) Int. Cl. C07C 69/52

(52) U.S. Cl. 560/224

(57) ABSTRACT

(Meth)acrylates of polyhydric alcohols are prepared by reacting (meth)acrylic acid and the corresponding polyhydric alcohol in the presence of at least one acidic catalyst and, if required, at least one polymerization inhibitor and in the presence or absence of a solvent, by a process in which the polyhydric alcohol used contains less than 500 ppm of bound formaldehyde.

The present invention describes a process for the preparation of (meth)acrylates of polyhydric alcohols by esterifying (meth)acrylic acid with the corresponding polyhydric alcohols in the presence of at least one acidic catalyst, if required a polymerization inhibitor/polymerization inhibitor mixture and, if required, a solvent which forms an azeotropic mixture with water.

Polyhydric alcohols are understood as meaning those compounds which have a plurality of hydroxyl groups, for example 2 to 6, preferably 2 to 4, particularly preferably 2 or 3, in particular 3.

Owing to their reactive double bonds, such (meth)acrylates are useful monomers which are used, for example, as coating raw materials for electron beam curing or as a component of UV-curable printing inks, surface coatings or molding or casting materials or in adhesives.

In particular, colorless products without natural odor and with a low acid number, advantageous viscosity properties and a long shelf life are required for these applications.

The preparation of (meth)acrylates by acid-catalyzed esterification of (meth)acrylic acid with the corresponding alcohols in the presence of an inhibitor/inhibitor system and, if required, a solvent, e.g. benzene, toluene or cyclohexane, is generally known.

Catalysts used are as a rule sulfuric acid, arylsulfonic acids or alkanesulfonic acids or mixtures thereof.

Since the formation of the ester of (meth)acrylic acid and alcohol is known to be based on an equilibrium reaction, as a rule one starting material is used in excess and/or the resulting water of esterification and/or the desired ester is removed from the equilibrium, in order to obtain cost-effective conversions. However, influencing the equilibrium of the esterification by the use of an excess of alcohol is disadvantageous since, inter alia, the formation of ethers from the starting alcohols and Michael adducts is promoted thereby (cf. for example U.S. Pat. No. 4,280,010, column 1).

Michael adducts are understood as meaning the products which are formed by an addition reaction of alcohols or (meth)acrylic acid at the double bond of (meth)acrylic compounds, for example alkoxypropionic acids or acrylyloxypropionic acids, and to their esters.

Since, owing to their high boiling points, the (meth)acrylates of polyhydric alcohols cannot as a rule be purified by distillation, these byproducts remain in the desired ester and influence the further processing and/or the quality of both the desired ester and the subsequent products.

In the preparation of higher (meth)acrylates, as a rule the water of reaction is therefore removed and in general an excess of (meth)acrylic acid is used. The water of esterification is usually separated off by distillation, by stripping, for example with air, or with the aid of a solvent which forms an azeotropic mixture with water.

Since (meth)acrylic compounds generally, and in particular polyfunctional (meth)acrylates, readily tend to undergo undesired polymerization, particularly under the action of heat, considerable efforts are generally made to avoid the formation of polymer during the esterification and the isolation of the desired ester.

In fact, this polymer formation generally leads to coating of the reactor walls, heat exchanger surfaces and column trays (fouling) and to blockage of pipes, pumps, valves, etc. (EP-A 522 709, page 2, lines 9-18; U.S. Pat. No. 5,171,888, column 1, lines 19-38). This results in expensive shutdowns and complicated cleaning operations, for example the boiling with basic solutions, which then have to be disposed of by an expensive procedure, as described in DE-A 195 36 179.

The polymer in the reaction mixture moreover hinders the working-up by causing phase separation problems during the washing of the reaction mixture. Since, as stated above, the higher (meth)acrylates of polyhydric alcohols cannot be purified by distillation, this polymer remains in the desired ester and influences the further processing and the quality of the polymers or copolymers prepared (U.S. Pat. No. 3,639,459, column 1, lines 40-55).

In order substantially to prevent the undesired polymer formation, the use of polymerization inhibitors or inhibitor systems is generally recommended.

U.S. Pat. No. 4,187,383 describes a process for esterifying (meth)acrylic acid with organic polyols at a reaction temperature of from 20 to 80° C. in the presence of from 50 to 5 000 ppm of an alkoxy-substituted phenol or alkylated alkoxyphenol as a polymerization inhibitor, by means of which process products having a color number of 4.0 Gardner or less is obtained. In example 1 of said publication, the polyol and a solvent are initially taken, acrylic acid, polymerization inhibitor and catalyst are added during the heating phase, washing is effected with 15% strength sodium hydroxide solution after the reaction and the solvent is stripped. The reaction times are up to 35 hours, the Gardner color numbers are at best <1, mechanical stirring is required and a technically simpler circulation evaporator (see below) cannot be used. 1.0 Gardner corresponds here to about 160 APHA and 4.0 Gardner correspond to about 800 APHA, so that the color numbers achievable by means of this process are not satisfactory.

In general, the reaction mixture obtained in the esterification substantially comprises the desired ester, the esterification catalyst, the inhibitors, the excess (meth)acrylic acid, possibly a solvent and higher molecular weight byproducts (for example polymer, ether and Michael adducts).

Catalysts, excess (meth)acrylic acid and, if required, parts of the inhibitors are separated off as a rule by treatment with aqueous bases, for example alkali solutions, and/or salt solutions (DE-A 198 36 788) or solid oxides, carbonates or hydroxides in powder form (DE-A 39 39 163, EP 449 919, EP 449 918, DE-A 1 493 004) or ion exchangers.

During these cleaning operations, the byproducts have an adverse effect in that they complicate or even prevent the phase separation in the individual wash steps.

Separating off any solvent present for removing the water of reaction is usually effected by distillation.

Since some of the (meth)acrylates of polyhydric alcohols are used in the coating sector, the color plays a major role, in addition to the purity and viscosity.

Since these (meth)acrylates cannot be purified by distillation, various measures have been proposed for obtaining colorless end products.

DE-A 38 43 938 proposes the addition of active carbon as early as during the esterification in order to prevent the formation of discolored reaction products (column 2, lines 63-68). If discolorations nevertheless occur, an additional treatment with a suitable decolorizing agent, e.g. alumina, is recommended (column 5, lines 20-27).

EP-A 995 738 recommends carrying out the esterification in the presence of supercritical carbon dioxide in order, inter alia, to prevent discolorations.

The known processes have the disadvantage that they are technically complicated and/or require complicated apparatuses and/or additional assistants and are unsuitable on an industrial scale.

It is an object of the present invention to provide an economical process which permits the preparation of low-viscosity (meth)acrylates of polyhydric alcohols in high purity and high yield in a simple manner and without additional assistants on an industrial scale.

We have found that this object is achieved by a process for the preparation of (meth)acrylates of polyhydric alcohols by reacting (meth)acrylic acid and the corresponding polyhydric alcohol in the presence of at least one acidic catalyst and, if required, at least one polymerization inhibitor and in the presence or absence of a solvent, wherein the polyhydric alcohol used contains less than 500 ppm of bound formaldehyde.

The term (meth)acrylic acid is used here for acrylic acid and methacrylic acid.

The process found has the following advantages:
1. During treatment with aqueous phases, organic and aqueous phases exhibit better separation
2. The end product is substantially colorless
3. High space-time yields are obtained since the phase separation is improved and hence smaller apparatuses are sufficient
4. The end products have a low viscosity

Polyhydric alcohols used are, for example, trimethylolbutane, trimethylolpropane, trimethylolethane, neopentyl glycol, pentaerythritol, 2-ethyl-1,3-propanediol, 2-methyl-1,3-propanediol, glycerol, ditrimethylolpropane, dipentaerythritol, bisphenol A, bisphenol F, bisphenol B, bisphenol S, 2,2-bis(4-hydroxycyclohexyl)propane, 1,1-, 1,2-, 1,3- and 1,4-cyclohexanedimethanol, 1,2-, 1,3- or 1,4-cyclohexanediol, sorbitol, mannitol, diglycerol, threitol, erythritol, adonitol (ribitol), arabitol (lyxitol), xylitol or dulcitol (galactitol).

Those polyhydric alcohols which are obtained by reacting an aldehyde with formaldehyde and then converting the aldehyde group into a hydroxyl group are preferably used in the novel process.

These are, for example, polyhydric alcohols of the formula (I):

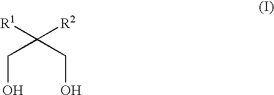

In this formula,
R¹ and R², independently of one another, are hydrogen, C₁-C₁₀-alkyl, C₁-C₁₀-hydroxyalkyl carboxyl or C₁-C₄-alkoxycarbonyl, preferably hydrogen, hydroxymethyl or C₁-C₁₀-alkyl, particularly preferably hydroxymethyl or C₁-C₁₀-alkyl.

The alkyl radicals may in each case be straight-chain or branched.

Examples of R¹ and R² are hydrogen, methyl, ethyl, isopropyl, n-propyl, n-butyl, isobutyl, sec-butyl, tert-butyl, n-pentyl, n-hexyl, n-heptyl, n-octyl, n-decyl, hydroxymethyl, carboxyl, methoxycarbonyl, ethoxycarbonyl and n-butyoxycarbonyl, preferably hydrogen, hydroxymethyl, methyl and ethyl, particularly preferably hydroxymethyl, methyl and ethyl.

Examples of polyhydric alcohols of the formula (I) are trimethylolbutane, trimethylolpropane, trimethylolethane, neopentylglycol, pentaerythritol, 2-ethyl-1,3-propanediol, 2-methyl-1,3-propanediol, 1,3-propanediol, dimethylolpropionic acid, methyl dimethylolpropionate, ethyl dimethylolpropionate, dimethylolbutyric acid, methyl dimethylolbutyrate and ethyl dimethylolbutyrate, preferably neopentylglycol, trimethylolpropane, pentaerythritol and dimethylolpropionic acid, particularly preferably neopentylglycol, trimethylolpropane and pentaerythritol, very particularly preferably trimethylolpropane and pentaerythritol, in particular trimethylolpropane.

Such polyhydric alcohols of the formula (I) are obtainable, for example, by reacting an aldehyde of the formula (II), 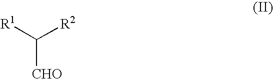
where R¹ and R² have the above meanings, with formaldehyde and then converting the aldehyde group into a hydroxyl group.

These polyhydric alcohols (I) are obtained on an industrial scale by condensation or formaldehyde with higher, CH-acidic aldehydes (II) or with water and acrolein or 2-alkylacroleins. In this reaction, a distinction is made between two fundamental procedures for converting the aldehyde group into a hydroxyl group, which is to be illustrated below for the preparation of trimethylolpropane but by no means restricted thereto.

On the one hand, there is the Cannizzaro process, which in turn is divided into the inorganic and the organic Cannizzaro process. In the inorganic variant, an excess of formaldehyde is reacted with the corresponding aldehyde (II), i.e. n-butyraldehyde, in the presence of stoichiometric amounts of an inorganic base, such as NaOH or Ca(OH)₂. The dimethylolbutanal formed in the first stage undergoes a disproportionation reaction with the excess formaldehyde in the second stage to give trimethylolpropane and the formate of the corresponding base, for example to give sodium formate or calcium formate. The production of these salts is a disadvantage since they are difficult to separate from the reaction product, and moreover one equivalent of formaldehyde is lost.

In the organic Cannizzaro process, a tertiary alkylamine is used instead of an inorganic base. This makes it possible to achieve higher yields than with an inorganic base. Trialkylammonium formate is obtained as an undesired byproduct. Thus, one equivalent of formaldehyde is lost here too.

The disadvantages of the Cannizzaro process are avoided in the hydrogenation process. There, formaldehyde is reacted with the corresponding aldehyde (II) in the presence of catalytic amounts of an amine. This ensures that the reaction stops substantially at the stage of the alkylolated aldehyde. After the formaldehyde has been separated off, the reaction mixture, which still contains small amounts of the corresponding polyhydric alcohol and of acetals of the alcohols formed, in addition to the alkylolated aldehyde mentioned, is subjected to a catalytic hydrogenation in which the desired polyhydric alcohol is obtained.

A particularly effective process for the preparation of polyhydric alcohols obtainable by condensation of aldehydes with formaldehyde is described in WO 98/28253. High yields in combination with the production of only small amounts of coupled products are permitted by this process. In this process, the higher aldehyde is reacted with from 2 to 8 times the amount of formaldehyde in the presence of a tertiary amine, and the reaction mixture thus obtained is separated into two solutions, one solution containing the completely methylolated alkanal mentioned and the other solution containing unconverted starting material. The latter solution is recycled into the reaction. The separation is effected by distillation or simple separation of the aqueous phase from the organic phase. The product-containing solution is subjected to a catalytic and/or thermal treatment in order to convert incompletely alkylolated alkanals into the desired completely methylolated compounds. Byproduct formed here is separated off by distillation, and the bottom product thus obtained is subjected to the catalytic hydrogenation, which leads to the polyhydric alcohols.

Particularly preferably used in the novel process for the preparation of (meth)acrylates are polyhydric alcohols of the formula (I) which have been obtained by the hydrogenation process, i.e. by reacting an aldehyde of the formula (II) with formaldehyde and then converting the aldehyde group into a hydroxyl group by catalytic hydrogenation, particularly preferably those which have been obtained by the process described in WO 98/28253.

According to the invention, it is essential that the content of bound formaldehyde in the polyhydric alcohol used is less than 500, preferably less than 400, ppm by weight.

Formaldehyde-containing acetals (formaldehydeacetals, formals) are those cyclic or aliphatic compounds which contain the structural element
—O—CH₂—O— (formula III).

These may be those hemiacetals or full acetals which are derived from main components and impurities, and byproducts, intermediate products or subsequent products of the reaction mixture.

These may be, for example, the following formaldehyde-containing acetals of the formula (IV):

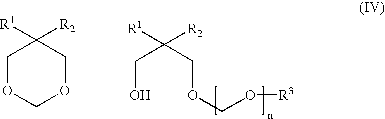

In this formula, R¹ and R² have the abovementioned meanings and furthermore
R³ is straight-chain or branched C₁-C₁₀-alkyl, preferably C₁-C₈-alkyl, particularly preferably C₁-C₅-alkyl, straight-chain or branched C₁-C₁₀-hydroxyalkyl, preferably C₁-C₈-hydroxyalkyl, particularly preferably C₁-C₆-hydroxyalkyl, or hydrogen and
n is an integer from 1 to 4, preferably from 1 to 3, particularly preferably 1 or 2.

Examples of R³ are hydrogen, methyl, ethyl, n-propyl, n-butyl, 2-methylpropyl, 2-methylbutyl, 2-ethyl-3-hydroxypropyl, 2-methyl-3-hydroxypropyl, 2,2-bis(hydroxymethyl)butyl, 2,2-bis(hydroxymethyl)propyl, 2,2-dimethyl-3-hydroxypropyl, 3-hydroxypropyl, 3-hydroxy-2-(hydroxymethyl)propyl or 3-hydroxy-2,2-bis(hydroxymethyl)propyl.

The following formaldehyde-containing acetals are preferred:

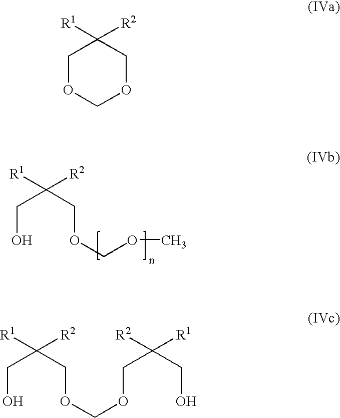

In these formulae, R¹, R² and n have the abovementioned meanings.

The formaldehyde-containing acetals IVa, IVb (n=1), Ivb (n=2) and IVc are particularly preferred.

The methanolacetals form from methanol which is generally contained in a small proportion in formaldehyde or forms in small amounts from formaldehyde during the preparation by Cannizzaro reaction.

Typical formaldehyde-containing acetals are, for example, for the case of the synthesis of the trihydric alcohol trimethylolpropane (TMP) from formaldehyde and n-butyraldehyde in the presence of catalytic amounts of trialkylamine, are the formaldehyde-containing acetals IVa, IVb (n=1), IVb (n=2) and IVc, where in each case R¹ is ethyl and R² is hydroxymethyl, which may be contained in an amount of from 0.05 to 10% by weight in the crude product of the hydrogenation process.

The content of bound formaldehyde is calculated as the total molecular weight fraction of formaldehyde equivalents in the respective formaldehyde-containing acetal multiplied by its analytically found weight fraction in the reaction mixture.

For example, the content of bound formaldehyde for a trimethylolpropane mixture (R¹=ethyl, R²=hydroxymethyl) which contains the components (IVa), (IVb, where n=1 and n=2) and (IVc) is calculated as follows:

$$\begin{matrix}Content of bound formaldehyde [ \% by weight ] = \\ \% by weight of ( IVa ) \times \frac{30 g \text{/} mol}{146 g \text{/} mol} + \\ \% by weight of ( IVb , n = 1 ) \times \frac{30 g \text{/} mol}{178 g \text{/} mol} + \\ \% by weight of ( IVb , n = 2 ) \times \frac{2 \times 30 g \text{/} mol}{208 g \text{/} mol} + \\ \% by weight of ( IVc ) \times \frac{30 g \text{/} mol}{280 g \text{/} mol}\end{matrix}$$

In order to obtain the corresponding content of bound formaldehyde in ppm by weight, this value should be multiplied by 10 000.

The content of the respective components can be determined by analytical methods known per se to a person skilled in the art, for example by gas chromatography or HPLC. The respective components can be identified, for example, by coupling said analytical methods with mass spectrometry.

It is not relevant according to the invention how such a low content of bound formaldehyde is achieved in the polyhydric alcohol.

U.S. Pat. No. 6,096,905 discloses a process in which a composition containing formaldehyde-containing acetals is treated with a strongly acidic catalyst at from 30 to 300° C. for from ½ to 8 hours.

GB-A 1 290 036 describes a process in which a crude TMP solution obtained by the inorganic Cannizzaro process is treated with a cation exchanger.

A preferred process for reducing the content of bound formaldehyde in a polyhydric alcohol comprises purifying the polyhydric alcohol by distillation after its preparation, then subjecting it to a heat treatment and then purifying it again, preferably by distillation, as described in DE-A 100 29 055 or in the International Application with the title Verfahren zum Enfernen von formaldehydhaltigen Acetalen aus mehrwertigen Alkoholen durch Tempern [Process for removing formaldehyde-containing acetals from polyhydric alcohols by heating] of BASF AG and the same date of filing as the present document.

If polyhydric alcohols are used in such a heating step, particularly good results can be obtained with the use of alcohol solutions containing more than 60%, preferably >75%, particularly preferably >90%, very particularly preferably >95%, in particular >98%. As further components, the alcohol solutions may contain, for example, solvents, such as water, methanol, ethanol or n-butanol, and byproducts occurring in the preparation of the polyhydric alcohol, preferably in amounts of less than 10, particularly preferably less than 5, very particularly preferably less than 2,% by weight.

This process can be used for reducing the content of bound formaldehyde in polyhydric alcohols, preferably those alcohols of the formula (I), in particular trimethylolpropane, of any origin. It is possible to use batches which originate from the organic or the inorganic Cannizzaro process. The best results were obtained when alcohols which originate from the hydrogenation process were used in the process serving for the reduction of the formaldehyde-containing acetals. In any case, it is important for the alcohol to be purified beforehand and to have a purity which is in the abovementioned range.

If it is intended to use the process to remove formaldehyde-containing acetals from crude solutions of polyhydric alcohols, in particular trimethylolpropane, having product contents of from 60 to 95% by weight, the crude product obtained by the hydrogenation process (the hydrogenation discharge) is preferably subjected, prior to the heating step, to dewatering in which the water and other low boilers, such as methanol and trialkylamine or trialkylammonium formate, are separated off by distillation.

In order to achieve the desired reduction in the content of bound formaldehyde in this process, it is necessary to maintain specific reaction conditions which may vary as a function of, for example, the type of polyhydric alcohol used, the purities of the products used, the apparatuses used and any further components or additives present. These reaction conditions are accessible to a person skilled in the art by means of experiments.

In general, the heating step is carried out at from 100 to 300° C., preferably from 160 to 240° C., with residence times of from 5 minutes to 24 hours, preferably from 15 minutes to 4 hours, and pressures of from 100 mbar to 200 bar, preferably from 1 to 10 bar.

If the polyhydric alcohol to be purified is trimethylolpropane, the heating step is carried out at from 100 to 300° C., preferably from 160 to 240° C., with residence times of from 5 minutes to 24 hours, preferably from 1 to 5 hours, particularly preferably from 15 minutes to 4 hours, and the abovementioned pressures.

The conventional apparatuses known to a person skilled in the art may be used for carrying out the heating step, it being possible for said heating step to be carried out continuously or batchwise. In the batchwise procedure, the heating step is preferably carried out in a stirred container; in the continuous procedure, it is preferably carried out in a tubular reactor, it being possible to employ a liquid-phase or trickle-bed method.

The most preferred embodiment of the heating step is the continuous procedure in a tubular reactor by the liquid-phase method.

In all these procedures, the reaction container can be provided with the conventional dumped packings known to a person skilled in the art, for example Raschig or Pall rings, or stacked packings, such as sheet metal packings, in order to achieve better mixing of the components. Supports and/or catalysts in the conventional finished forms, for example extrudates or pellets, ay also be present in order to accelerate the reactions taking lace in the heating step. Examples of suitable supports/catalysts include TiO₂, Al₂O₃, SiO₂, supported phosphoric acid (H₃PO₄) and zeolites.

In one variant of the heating step, a suitable additive is added to the reaction solution during the heating step, in order to accelerate or to facilitate the reactions leading to a reduction in the amount of the formaldehyde-containing acetals. Acids which are not too strong and/or have a reducing effect or anhydrides thereof or ion exchangers, as described in U.S. Pat. No. 6,096,905 or GB 1 290 036 are suitable for this purpose. Examples of suitable acids include phosphoric acid, phosphorous acid, hypophosphorous acid, boric acid, carbonic acid and sulfurous acid. Gases such as CO₂ and SO₂, which are acidic in aqueous solution, are also suitable.

The acids to be used as an additive are employed in amounts of from 10 ppm to 1% by weight, preferably from 100 to 2 000 ppm. Since any assistant added has to be separated from the reduced-formaldehydeacetal polyhydric alcohol after the heating step, it is preferable if this additive is gaseous and can therefore be removed in a simple manner by devolatilization of the reaction mixture.

It may furthermore be advantageous to carry out the heating step for decomposing the formaldehyde-containing acetals in the presence of an inert gas, for example nitrogen, argon or helium, preferably under nitrogen.

Without being tied to a theory, it is presumed that formaldehyde-containing acetals are converted by the heating step, in the alcohol prepurified by distillation, into higher-boiling, sparingly volatile components and low-boiling components and can thus be more readily separated off by distillation.

The polyhydric alcohol having a reduced content of bound formaldehyde can be readily separated from the resulting high-boiling sparingly volatile components by distillation. The heating step is therefore generally followed by a distillation. Since the sparingly volatile compounds formed in the heating step from the formaldehyde-containing acetals generally differ substantially from the polyhydric alcohols with respect to their boiling behavior, they can be separated off by simple distillative measures or methods having only a small separation effect. Separation units comprising only one distillation stage, for example falling-film evaporators or thin-film evaporators, are often sufficient. If necessary, particularly if the distillation also serves for further purification of the product alcohol, more complicated separation methods or separation apparatuses are used, generally columns having a plurality of theoretical stages, for example columns containing dumped packings, bubble-tray columns or columns containing stacked packings.

In the distillation, the conventional pressure and temperature conditions known to a person skilled in the art are maintained, said conditions of course also being dependent on the product alcohol used.

According to a further embodiment, the heating step can also be combined with the distillation. In this case, the heating takes place in the bottom of a column of the distillation apparatus, in which the polyhydric product alcohol is separated from the sparingly volatile components formed during the heating and any other impurities. If heating step and distillation are combined in one stage, it is important that the reaction conditions described above and relating to pressure, temperature and in particular the residence time are maintained, in order to achieve sufficient decomposition of the formaldehyde-containing acetals. When heating step and distillation step are combined to give a single process step, the addition of acid is preferred.

The polyhydric alcohol obtainable by this process generally has a content of bound formaldehyde, as defined above, of less than 500, preferably less than 400, ppm by weight.

The process by which the polyhydric alcohol was obtained, for example by the Cannizzaro or the hydrogenation process, is unimportant.

In the case of trimethylolpropane, for example, the following components may also be present:

| | |
|---|---|
| Trimethylolpropane | 97.0-99.95% |
| 2-Methylbutan-1-ol | up to 3%, e.g. 10 ppm - 2% |
| 2-Ethyl-1,3-propanediol | up to 3%, e.g. 10 ppm - 2% |
| Trimethylolpropane mono-, | up to 3%, e.g. 10 ppm - 2% |
| -di or triformate (total) | |
| Di-Trimethylolpropane | up to 3%, e.g. 10 ppm - 2% |
| Higher condensates | up to 3%, e.g. 10 ppm - 2% |

Higher condensates may be, for example, dimethylolbutanal-trimethylolpropaneacetal, monomethylolbutanal-trimethylolpropaneacetal or ethylacrolein-trimethylolacetal.

Alkoxylated alcohols which are obtainable by reacting a polyhydric alcohol containing less than 500, preferably less than 400, ppm of bound formaldehyde with at least one alkylene oxide are furthermore suitable as alcohols for the esterification with (meth)acrylic acid.

Suitable alkylene oxides are, for example, ethylene oxide, propylene oxide, isobutylene oxide, vinyloxirane and/or styrene oxide, preferably ethylene oxide, propylene oxide and/or isobutylene oxide, particularly preferably ethylene oxide and/or propylene oxide.

Preferred examples of such alkoxylated alcohols are the alkoxylation products (Va), (Vb) or (Vc) of alcohols of the formula (I) 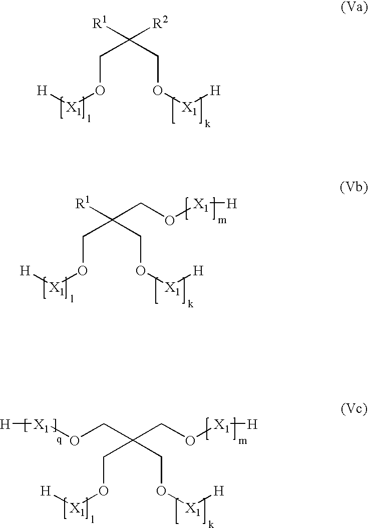
where
R¹ and R² have the abovementioned meanings,
k, l, m and q are each an integer from 1 to 30, preferably from 1 to 20, particularly preferably from 1 to 10, in particular from 1 to 5, and
each Xᵢ for i=1 to k, 1 to l, 1 to m and 1 to q can, independently of one another, be selected from the group consisting of —CH₂—CH₂—O—, —CH₂—CH(CH₃)—O—, —CH(CH₃)—CH₂—O—, —CH₂—C(CH₃)₂—O—, —C(CH₃)₂—CH₂—O—, —CH₂—CHVin-O—, —CHVin—CH₂—O—, —CH₂—CHPh-O— and —CHPh-CH₂—O—, preferably from the group consisting of —CH₂—CH₂—O—, —CH₂—CH(CH₃)—O— and —CH(CH₃)—CH₂—O—,
where Ph is phenyl and Vin is vinyl.

These are preferably mono- to deca-, particularly preferably mono- to pentaethoxylated, propoxylated or mixed ethoxylated and propoxylated neopentylglycol, trimethylolpropane, trimethylolethane or pentaerythritol.

Among these, those polyhydric alcohols of the formula (Vb) are particularly preferred.

If mixed alkoxylated alcohols are used, the different alkoxy groups contained therein may be present in a molar ratio to one another of, for example, 0.05-20:1, preferably 0.1-10:1, particularly preferably 0.2-5:1.

The reaction of the alcohols with an alkylene oxide is known per se to a person skilled in the art. Possible procedures are described in Houben-Weyl, Methoden der Organischen Chemie, 4th Edition, 1979, Thieme Verlag Stuttgart, Editor Heinz Kropf, Volume 6/1a, Part 1, pages 373 to 385.

The reaction is preferably carried out as follows:

The polyhydric alcohol, if necessary dissolved in a suitable solvent, e.g. benzene, toluene, xylene, tetrahydrofuran, hexane, pentane or petroleum ether, is initially taken at from 0 to 120° C., preferably from 10 to 100° C., particularly preferably from 20 to 80° C., preferably under an inert gas, e.g. nitrogen. The alkylene oxide, if necessary dissolved at from −30 to 50° C. in one of the abovementioned solvents is metered in continuously or in portions with thorough mixing, so that the temperature of the reaction mixture is kept at from 120 to 180° C., preferably from 120 to 150° C. The reaction may take place at up to 60, preferably up to 30, particularly preferably up to 10, bar.

The amount of alkylene oxide is adjusted so that up to (1.1×(k+l+m+q)), preferably up to (1.05×(k+l+m+q)), particularly preferably (k+l+m+q), mol of alkylene oxide are metered in per mol of polyhydric alcohol, where k, l, m and q have the abovemetioned meanings.

If required, up to 50, particularly preferably up to 25, very particularly preferably up to 10, mol %, based on the polyhydric alcohol, of a catalyst can be added for acceleration, for example water, monoethanolamine, diethanolamine, triethanolamine, dimethylaminoethanolamine, ethylene glycol or diethylene glycol, and alkali metal hydroxides, alcoholates or hydrotalcite, preferably alkali metal hydroxides in water.

After the alkylene oxide has been completely metered in, the reaction is generally allowed to continue for from 10 to 500, preferably from 20 to 300, particularly preferably from 30 to 180, minutes at from 30 to 220° C., preferably from 80 to 200° C., particularly preferably from 100 to 180° C., it being possible for the temperature to remain constant or to be increased stepwise or continuously.

The conversion of alkylene oxide is preferably at least 90%, particularly preferably at least 95%, very particularly preferably at least 98%. Any residues of alkylene oxide can be stripped out by passing a gas, for example nitrogen, helium, argon or steam, through the reaction mixture.

The reaction can be carried out, for example, batchwise, semicontinuously or continuously in a stirred reactor or continuously in a tubular reactor with static mixers.

The reaction is preferably carried out completely in the liquid phase.

The reaction product formed can be further processed in crude or worked-up form.

If further use in pure form is desired, the product can be purified, for example by crystallization and solid/liquid separation.

The yields are as a rule more than 75%, generally more than 80%, frequently more than 90%.

Those polyetherols, obtainable by a process for the preparation of polyetherols, as described in the German patent application with the application number 10223054.4 of May 24, 2002, are furthermore preferred.

Moreover, polyesterols can advantageously be used in the novel preparation of (meth)acrylates, as described in the German patent application with the application number 10223055.2 of May 24, 2002. Such polyesterols are obtainable by reacting polyhydric alcohols with at least one dicarboxylic acid and/or one derivative, for example an anhydride of a dicarboxylic acid, if required in a solvent, with removal of the water of reaction, the polyhydric alcohol used having a formaldehyde acetal content of less than 500 ppm.

Polyhydric alcohols are those as described in this document.

For the preparation of such polyesterpolyols, the acids used may be carboxylic acids having at least two acid groups, preferably aliphatic or aromatic dicarboxylic acids, in particular those of 2 to 12 carbon atoms. Examples of suitable dicarboxylic acids are: adipic acid, succinic acid, glutaric acid, suberic acid, azelaic acid, sebacic acid, decanedicarboxylic acid, maleic acid, fumaric acid, dimeric and/or trimeric fatty acids and preferably adipic acid, phthalic acid, isophthalic acid, terephthalic acid and the isomeric naphthalenedicarboxylic acids. The dicarboxylic acids may be used individually or as a mixture with one another. Instead of the free dicarboxylic acids, it is also possible to use the corresponding dicarboxylic acid derivatives, for example dicarboxylic esters of alcohols of 1 to 4 carbon atoms or dicarboxylic anhydrides. Dicarboxylic acid mixtures comprising succinic, glutaric and adipic acid in weight ratios of, for example, 20 to 35:35 to 50:20 to 32% and adipic acid and in particular mixtures of phthalic acid and/or phthalic anhydride and adipic acid, mixtures of phthalic acid (anhydride), isophthalic acid and adipic acid or dicarboxylic acid mixtures comprising succinic, glutaric and adipic acid and mixtures of terephthalic acid and adipic acid or dicarboxylic acid mixtures comprising succinic, glutaric and adipic acid are preferably used. For use in rigid polyurethane foams, aromatic carboxylic acids or mixtures which contain aromatic carboxylic acids are preferably used. Furthermore, fatty acids and their derivatives, including dimeric and/or trimeric fatty acids, are concomitantly used individually or as a mixture. Polyester alcohols based on long-chain carboxylic acids, in particular fatty acids, can preferably be used for the preparation of alkyd resins, which may be further processed to give finishes.

The alcohols of the formula (I) can be used as a mixture with further polyhydric alcohols, preferably diols, of 2 to 12, preferably 2 to 6, carbon atoms. Examples of di- and polyhydric alcohols, in particular diols, are: ethanediol, diethylene glycol, 1,2- and 1,3-propanediol, dipropylene glycol, 1,4-butanediol, 1,5-pentanediol, 1,6-hexanediol, 1,10-decanediol, glycerol and trimethylolpropane. Ethanediol, diethylene glycol, 1,4-butanediol, 1,5-pentanediol, 1,6-hexanediol or mixtures of at least two of said diols, in particular mixtures of 1,4-butanediol, 1,5-pentanediol and 1,6-hexanediol, are preferably used. Polyesterpolyols obtained from lactones, e.g. ε-caprolactone, or hydroxycarboxylic acids, e.g. ω-hydroxycaproic acid and hydroxybenzoic acids, can furthermore be used.

If required, monofunctional alcohols and/or carboxylic acids can also be used as a mixture with the polyfunctional alcohols and carboxylic acids for establishing the functionality. Examples of monofunctional carboxylic acids are monomeric fatty acids, for example oleic acid or ricinoleic acid. Examples of monomeric alcohols are aliphatic alcohols of 1 to 15, preferably 2 to 10, carbon atoms, for example hexanol, octanol, nonanol or decanol.

The alcohols of the formula (I) are preferably used in an amount of not more than 50% by weight, based on the weight of the polyester alcohol, in particular if they have a functionality greater than 2, since otherwise undesirable crosslinking and, associated therewith, high viscosity of the polyester alcohols occur.

For the preparation of the polyesterpolyols, the organic, e.g. aliphatic and preferably aromatic, carboxylic acids and mixtures of aromatic and aliphatic polycarboxylic acids and/or derivatives thereof and polyhydric alcohols can be subjected to polycondensation in the absence of a catalyst or, preferably, in the presence of esterification catalysts, expediently in an atmosphere comprising inert gases, e.g. nitrogen, carbon monoxide, helium, argon, etc., in the melt at from 150 to 250° C., preferably from 180 to 220° C., under atmospheric or reduced pressure, to the desired acid number, which is advantageously less than 10, preferably less than 2. For the preparation of the polyesterpolyols, the organic polycarboxylic acids and/or derivatives thereof and polyhydric alcohols are advantageously subjected to polycondensation in a molar ratio of 1:1 to 1.8, preferably 1:1.05 to 1.2.

Catalysts used may be acidic catalysts, such as toluenesulfonic acids, preferably organometallic compounds, in particular those based on titanium or tin, such as titanium tetrabutylate or tin(II) octanoate.

It is also possible first to react the polyhydric alcohol with alkylene oxides to give a polyether alcohol and to esterify this with carboxylic acids. It is likewise possible to add an alkoxylation catalyst to the prepared polyester alcohol and to subject the latter to an addition reaction with alkylene oxide. The polyetheresterols thus obtained have good compatibility with polyetherols and polyesterols and can preferably be used for the preparation of polyurethanes.

The process by which the preparation of the (meth)acrylate from (meth)acrylic acid and polyhydric alcohol is carried out is not limited. According to the invention, what is important is that the polyhydric alcohol used has a content of bound formaldehyde, as defined above, of less than 500, preferably less than 400, ppm by weight.

When such a polyhydric alcohol is used according to the invention in an esterification process, a person skilled in the art will discover the advantage which the use of this particular polyhydric alcohol has over a conventional polyhydric alcohol having a higher content of bound formaldehyde in the same esterification process.

The preparation and/or working-up processes known to a person skilled in the art for the polyhydric alcohols can be used for the esterification, for example those mentioned at the outset or those described in DE-A 199 41 136, DE-A 38 43 843, DE-A 38 43 854, DE-A 199 37 911, DE-A 199 29 258, EP-A 331 845, EP 554 651 or U.S. Pat. No. 4,187,383.

In general, the esterification can be carried out as follows:

The esterification apparatus consists of a stirred reactor, preferably of a reactor having a circulation evaporator and an attached distillation unit with condenser and phase separation vessel.

The reactor may be, for example, a reactor having double-jacket heating and/or internal heating coils. Preferably, a reactor having an external heat exchanger and natural or forced circulation, i.e. with the use of a pump, particularly preferably natural circulation, in which circulation is effected without mechanical aids, is used.

Of course, the reaction can also be carried out in a plurality of reaction zones, for example a reactor cascade comprising two to four, preferably two or three, reactors.

Suitable circulation evaporators are known to a person skilled in the art and are described, for example, in R. Billet, Verdampfertechnik, HTB-Verlag, Bibliographisches Institut Mannheim, 1965, 53. Examples of circulation evaporators are tube-bundle heat exchangers, plate-type heat exchangers, etc.

Of course, a plurality of heat exchangers may also be present in the circulation.

The distillation unit is of a design known per se. It may be a simple distillation unit which, if required, is equipped with a spray guard or it may be a rectification column. Suitable column internals are in principle all conventional internals, for example trays, stacked packings and/or dumped packings. Among the trays, bubble trays, sieve trays, valve trays, Thormann trays and/or dual-flow trays are preferred; among the dumped packings, those comprising rings, coils, saddle elements or braids are preferred.

As a rule, from 5 to 20 theoretical plates are sufficient.

The condenser and the separation vessel are of conventional design.

(Meth)acrylic acid and polyhydric alcohols are used, as a rule, in equivalent amounts, based on the hydroxyl groups of the alcohol, but it is also possible to use less than the stoichiometric amount or an excess of (meth)acrylic acid.

The molar ratio of the number of hydroxyl groups in the polyhydric alcohol to (meth)acrylic acid is in general 1:0.9-3, preferably 1:1.0-2.0, particularly preferably 1:1.05-1.5, very particularly preferably 1:1.05-1.25, in particular 1:1.05-1.1.

Suitable esterification catalysts are the conventional mineral acids and sulfonic acids, preferably sulfuric acid, phosphoric acid, alkanesulfonic acids (e.g. methanesulfonic acid, trifluoromethanesulfonic acid) and/or arylsulfonic acids (e.g. benzene-, p-toluene- or dodecylbenzenesulfonic acid) or mixtures thereof, but acidic ion exchangers are also possible.

Sulfuric acid, methanesulfonic acid and p-toluenesulfonic acid or mixtures thereof are particularly preferred.

They are used, as a rule, in an amount of 0.1-5, preferably 0.5-5, particularly preferably 1-4, very particularly preferably 2-4, % by weight, based on the esterification mixture.

If required, the esterification catalyst can be removed from the reaction mixture with the aid of an ion exchanger. The ion exchanger can be added directly to the reaction mixture and then filtered off or the reaction mixture can be passed over an ion exchanger bed.

Preferably, the esterification catalyst is left in the reaction mixture and removed subsequently by washing (see below).

Suitable polymerization inhibitors which can be used for the esterification are phenothiazine, monohydric and polyhydric phenols which, if required, have one or more alkyl groups, such as alkylphenols, for example o-, m- or p-cresol (methylphenol), 2-tert-butyl-4-methylphenol, 6-tert-butyl-2,4-dimethylphenol, 2,6-di-tert-butyl-4-methylphenol, 2-methylhydroquinone, 2,5-di-tert-butylhydroquinone, 2-tert-butylphenol, 4-tert-butylphenol, 2,4-di-tert-butylphenol, 2-methyl-4-tert-butylphenol, 4-tert-butyl-2,6-dimethylphenol, 2,5-di-tert-butylhydroquinone, toluhydroquinone or 2,2′-methylenebis(6-tert-butyl-4-methyl-phenol), hydroxyphenols, for example hydroquinone, pyrocatechol (1,2-dihydroxybenzene) or benzoquinone, aminophenols, e.g. para-aminophenol, nitrosophenols, e.g. para-nitrosophenol, alkoxyphenols, for example 2-methoxyphenol (guajacol, pyrocatechol monomethyl ether), 2-ethoxyphenol, 2-isopropoxy-phenol, 4-methoxyphenol (hydroquinone monomethyl ether), mono- or di-tert-butyl-4-methoxyphenol, tocopherols, e.g. α-tocopherol, and 2,3-dihydro-2,2-dimethyl-7-hydroxybenzofuran (2,2-dimethyl-7-hydroxycoumaran), phosphorus compounds, e.g. triphenyl phosphite, hypophosphorous acid or alkyl esters of phosphorous acid, copper or manganese, cerium, nickel, chromium or copper salts, for example the chlorides, sulfates, salicylates, tosylates, acrylates or acetates thereof, 4-hydroxy-2,2,6,6-tetramethylpiperidin-N-oxyl, 4-oxo-2,2,6,6-tetramethyl-piperidin-N-oxyl, 4-acetoxy-2,2,6,6-tetramethylpiperidin-N-oxyl, 2,2,6,6-tetramethyl-piperidin-N-oxyl, 4,4′,4″-tris-(2,2,6,6-tetramethylpiperidin-N-oxyl) phosphite or 3-oxo-2,2,5,5-tetramethylpyrrolidin-N-oxyl, N,N-diphenylamine, N-nitrosodiphenylamine, N,N′-dialkyl-para-phenylenediamines and mixtures thereof.

The esterification is preferably carried out in the presence of at least one polymerization inhibitor. At least one compound from the group consisting of phenothiazine, hydroquinone, hydroquinone monomethyl ether, 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, 4-tert-butyl-2,6-dimethylphenol, hypophosphorous acid, copper acetate, copper chloride and copper salicylate is particularly preferably used as the polymerization inhibitor (mixture).

For further supporting the stabilization, an oxygen-containing gas, preferably air or a mixture of air and nitrogen (air having a low oxygen content), may be present.

This oxygen-containing gas is preferably metered into the bottom region of a column and/or into a circulation evaporator and/or passed through the reaction mixture and/or over the latter.

The polymerization inhibitor (mixture) can be used in a total amount of 0.01-1, preferably 0.02-0.8, particularly preferably 0.05-0.5, % by weight, based on the esterification mixture.

The polymerization inhibitor (mixture) can be used, for example, as an aqueous solution or as a solution in a starting material or product.

The water of reaction formed in the reaction can be distilled off, it being possible to support this process by means of a solvent which forms an azeotropic mixture with water.

Suitable solvents for azeotropic removal of the water of reaction, if desired, are in particular aliphatic, cycloaliphatic and aromatic hydrocarbons or mixtures thereof.

n-Pentane, n-hexane, n-heptane, cyclohexane, methylcyclohexane, benzene, toluene or xylene is preferably used. Cyclohexane, methylcyclohexane and toluene are particularly preferred.

The esterification is preferably carried out in the presence of a solvent

The amount of solvent used is 10-200, preferably 20-100, particularly preferably 30-100, % by weight, based on the sum of alcohol and (meth)acrylic acid.

The reaction temperature of the esterification is in general 40-160° C., preferably 60-140° C., particularly preferably 80-120° C. The temperature may remain constant or increase in the course of the reaction; it is preferably increased in the course of the reaction. In this case, the final temperature of the esterification is 5-30° C. higher than the initial temperature. The temperature of the esterification can be determined and regulated by varying the solvent concentration in the reaction mixture, as described in DE-A 199 41 136 and the German Application with the application number 100 63 175.4.

If a solvent is used, it can be distilled off from the reaction mixture via the distillation unit attached to the reactor.

The distillate can either be removed or, after condensation, can be fed into a phase separation apparatus. The aqueous phase thus obtained is as a rule discharged and the organic phase can be fed as reflux into the distillation unit and/or passed directly into the reaction zone and/or fed into a circulation evaporator, as described in the German Patent Application with the application number 100 63 175.4.

When used as reflux, the organic phase can, as described in DE-A 199 41 136, be used for controlling the temperature in the esterification.

If the water contained in the reaction mixture is not removed via an azeotrope-forming solvent, it is possible to remove it by stripping with an inert gas, preferably an oxygen-containing gas, particularly preferably with air or air having a low oxygen content.

The esterification can be carried out at atmospheric, superatmospheric or reduced pressure and is preferably effected at atmospheric pressure.

The reaction time is as a rule 2-20, preferably 4-15, particularly preferably 7 to 12, hours.

According to the invention, the sequence of addition of the individual reaction components is not important. All components may be initially taken as a mixture and then heated or one or more components may be omitted or only partly initially taken and added only after the heating.

The (meth)acrylic acid which can be used is not limited and, in the case of crude (meth)acrylic acid, may contain, for example, the following components:

| | |
|---|---|
| (Meth)acrylic acid | 90-99.9% by weight |
| Acetic acid | 0.05-3% by weight |
| Propionic acid | 0.01-1% by weight |
| Diacrylic acid | 0.01-5% by weight |
| Water | 0.05-5% by weight |
| Aldehydes | 0.01-0.3% by weight |
| Inhibitors | 0.01-0.1% by weight |
| Maleic acid | 0.001-0.5% by weight |
| (anhydride) | |

The crude (meth)acrylic acid used is as a rule stabilized with 200-600 ppm of phenothiazine or other stabilizers in amounts which permit comparable stabilization.

Here, crude (meth)acrylic acid is understood as meaning the (meth)acrylic acid-containing mixture which is obtained after absorption of the reaction gases of the propane/propene/acrolein or isobutane/isobutene/methacrolein oxidation in an absorbent and subsequent removal of the absorbent, or which is obtained by fractional condensation of the reaction gases.

It is of course also possible to use pure (meth)acrylic acid having, for example, the following purity:

| | |
|---|---|
| (Meth)acrylic acid | 99.7-99.99% by weight |
| Acetic acid | 50-1 000 ppm by weight |
| Propionic acid | 10-500 ppm by weight |
| Diacrylic acid | 10-500 ppm by weight |
| Water | 50-1 000 ppm by weight |
| Aldehydes | 1-500 ppm by weight |
| Inhibitors | 1-300 ppm by weight |
| Maleic acid (anhydride) | 1-200 ppm by weight |

The pure (meth)acrylic acid used is as a rule stabilized with 100-300 ppm of hydroquinone monomethyl ether or other storage stabilizers in amounts which permit comparable stabilization.

Pure or prepurified (meth)acrylic acid is generally understood as meaning (meth)acrylic acid whose purity is at least 99.5% by weight and which is substantially free of the aldehydic, other carbonyl-containing and high-boiling components.

The aqueous phase of the condensate of the esterification reaction, which phase generally contains 0.1-10% by weight of (meth)acrylic acid, is separated off and discharged. Advantageously, the (meth)acrylic acid contained therein can be extracted with an extracting agent, preferably any solvent used in the esterification, for example with cyclohexane, at from 10 to 40° C. and a ratio of aqueous phase to extracting agent of 1:5-30, preferably 1:10-20, and can be recycled into the esterification.

For further supporting the circulation, an inert gas, preferably an oxygen-containing gas, particularly preferably air or a mixture of air and nitrogen (air having a low oxygen content) can be passed into the circulation, through or over the reaction mixture, for example in amounts of 0.1-1, preferably 0.2-0.8, particularly preferably 0.3-0.7, m³/m³h, based on the volume of the reaction mixture.

The course of the esterification can be monitored by monitoring the amount of water discharged and/or the decrease in the (meth)acrylic acid concentration in the reactor.

The reaction can be stopped, for example, as soon as 90%, preferably at least 95%, particularly preferably at least 98%, of the theoretically expected amount of water has been discharged by the solvent.

After the end of the esterification, the reactor mixture can be cooled in a conventional manner to a temperature of from 10 to 30° C. and the concentration of the desired ester can be brought to 60-80%, if required by adding solvent which may be the same as any solvent used for azeotropic removal of water or a different solvent.

If required, the reaction mixture may be subjected to a decolorization, for example by treatment with active carbon or metal oxides, e.g. alumina, silica, magnesium oxide, zirconium oxide, boron oxide or a mixture thereof, in amounts of, for example, 0.1-50, preferably 0.5-25, particularly preferably 1-10, % by weight at, for example, from 10 to 100° C., preferably from 20 to 80° C., particularly preferably from 30 to 60° C.

This can be effected by addition of the decolorizing agent in powder or granular form to the reaction mixture and subsequent filtration or by passing the reaction mixture over a bed of the decolorizing agent in the form of any desired, suitable moldings.

The decolorization of the reaction mixture can be effected at any desired point in the working-up process, for example at the stage of the crude reaction mixture or after any prewash, neutralization, wash or solvent removal.

The reaction mixture can furthermore be subjected to a prewash and/or a neutralization and/or a subsequent wash, preferably at least one neutralization and particularly preferably neutralization and subsequent wash. If required, neutralization and prewash can also be interchanged in the sequence.

(Meth)acrylic acid and/or catalyst present can be at least partly recovered from the aqueous phase of the washes and/or neutralization by acidification and extraction with a solvent and can be reused.

For the prewash or subsequent wash, the reaction mixture is treated in a wash apparatus with a wash liquid, for example water or a 5-30, preferably 5-20, particularly preferably 5-15, % strength by weight sodium chloride, potassium chloride, ammonium chloride, sodium sulfate or ammonium sulfate solution, preferably water or sodium chloride solution.

The ratio of reaction mixture to wash liquid is as a rule 1:0.1-1, preferably 1:0.2-0.8, particularly preferably 1:0.3-0.7.

The wash or neutralization can be carried out, for example, in a stirred container or in other conventional apparatuses, for example in a column or mixer-settler apparatus.

All extraction and wash processes and apparatuses known per se can be used for a wash or neutralization in the novel process, for example those which are described in Ullmann's Encyclopedia of Industrial Chemistry, 6th ed, 1999 Electronic Release, chapter entitled: Liquid—Liquid Extraction—Apparatus. For example, these may be one-stage or multistage, preferably one-stage, extractions and those by the cocurrent or countercurrent procedure, preferably the countercurrent procedure.

Sieve tray columns, columns having stacked or dumped packings, stirred containers or mixer-settler apparatuses and pulsed columns or those having rotating internals are preferably used.

The prewash is preferably used when metal salts, preferably copper salts, or preferably copper are (concomitantly) used as inhibitors.

Subsequent washing may be advantageous for removing traces of base or salt from the neutralized reaction mixture.

For neutralization, the reaction mixture which may have been prewashed and which may still contain small amounts of catalyst and the main amount of excess (meth)acrylic acid can be neutralized with a 5-25, preferably 5-20, particularly preferably 5-15, % by strength by weight aqueous solution of a base, for example an alkali metal or alkaline earth metal oxide, hydroxide, carbonate or bicarbonate, preferably sodium hydroxide solution, potassium hydroxide solution, sodium bicarbonate, sodium carbonate, potassium bicarbonate, calcium hydroxide, ammonia water or potassium carbonate, to which, if required, 5-15% by weight of sodium chloride, potassium chloride, ammonium chloride or ammonium sulfate may have been added, particularly preferably with sodium hydroxide solution or sodium hydroxide/sodium chloride solution.

The addition of the base is effected in a manner such that the temperature in the apparatus does not exceed 35° C. and is preferably from 20 to 35° C. and the pH is 10-14. The heat of neutralization is preferably removed by cooling the container with the aid of internal cooling coils or via double-jacket cooling.

The ratio of reaction mixture to neutralizing liquid is as a rule 1:0.1-1, preferably 1:0.2-0.8, particularly preferably 1:0.3-0.7.

The above statements are applicable with regard to the apparatus.

If a solvent is contained in the reaction mixture, it can be substantially removed by distillation and/or stripping. Any solvent present is preferably removed from the reaction mixture after washing and/or neutralization; if desired, however, this can also be effected before the wash or neutralization.

For this purpose, an amount of storage stabilizer, preferably hydroquinone monomethyl ether, such that, after removal of the solvent, 100-500, preferably 200-500, particularly preferably 200-400, ppm thereof are contained in the desired ester (residue) can be added to the reaction mixture.

The removal of the main amount of solvent by distillation is effected, for example, in a stirred kettle having double-jacket heating and/or internal heating coils under reduced pressure, for example at 20-700, preferably 30-500, particularly preferably 50-150, mbar and 40-80° C.

The distillation can of course also be carried out in a falling-film or thin-film evaporator. For this purpose, the reaction mixture is preferably circulated several times through the apparatus, under reduced pressure, for example at 20-700, preferably 30-500, particularly preferably 50-150, mbar and 40-80° C.

An inert gas, preferably an oxygen-containing gas, particularly preferably air or a mixture of air and nitrogen (air having a low oxygen content), can advantageously be passed into the distillation apparatus, for example 0.1-1, preferably 0.2-0.8, particularly preferably 0.3-0.7, m³/m³h, based on the volume of the reaction mixture.

The residual solvent content in the residue after the distillation is as a rule less than 5, preferably 0.5-5, particularly preferably 1-3, % by weight.

The solvent separated off is condensed and preferably reused.

If required, stripping by means of a solvent can be carried out in addition to or instead of the distillation.

For this purpose, the desired ester, which still contains small amounts of solvent, is heated to 50-80° C. and the remaining amounts of solvent are removed using a suitable gas in a suitable apparatus.

Suitable apparatuses are, for example, columns of a design known per se which have the conventional internals, for example trays, dumped packings or stacked packings, preferably dumped packings. In principle, all conventional internals are suitable as column internals, for example trays, stacked packings and/or dumped packings. Among the trays, bubble trays, sieve trays, valve trays, Thormann trays, and/or dual-flow trays are preferred; among the dumped packings, those comprising rings, coils, saddle elements, Raschig, Intos or Pall rings, barrel or Intalox saddles, Top-Pak, etc. or braids are preferred.

A falling-film, thin-film or wiped-film evaporator, for example a Luwa, Rotafilm or Sambay evaporator, which may be equipped, for example, with a demister as a spray guard, is also possible here.

Suitable gases are gases which are inert under the stripping conditions, preferably oxygen-containing gases, particularly preferably air or mixtures of air and nitrogen (air having a low oxygen content), in particular those which have been preheated to from 50 to 100° C.

The amount of stripping gas is, for example, 5-20, particularly preferably 10-20, very particularly preferably 10-15, m³/m³h, based on the volume of the reaction mixture.

If necessary, the ester can be subjected to a filtration at any stage of the working-up process, preferably after washing/neutralization and any solvent removal carried out, in order to remove precipitated traces of salt and any decolorizing agent present.

The (meth)acrylates of polyhydric alcohols, which (meth)acrylates are obtainable by the novel process, are clear and substantially colorless (color number, for example in the case of trimethylolpropane triacrylate, as a rule <150 APHA corresponds to Hazen) and have more advantageous color numbers, a lower viscosity (in the case of trimethylolpropane triacrylate, as a rule 160 mPa.s or less) and generally a lower polymer fraction than if polyhydric alcohols having a higher content of bound formaldehyde were used in the same process.

The present invention furthermore relates to a reaction mixture which substantially contains trimethylolpropane triacrylate and is obtainable by the novel process.

For this purpose, at least 10, preferably at least 20, particularly preferably at least 30, % by weight, based on the sum of trimethylolpropane and acrylic acid, of the solvent and at least a part of the polymerization inhibitor (mixture) are initially taken in a reactor having a circulation, preferably a natural circulation, and are heated and trimethylolpropane which has a content of bound formaldehyde, as defined above, of less than 500, preferably less than 400, ppm by weight and acrylic acid in a ratio of 1:3.3-4.5, preferably 1:3.6-4.5, particularly preferably 1:3.9-4.5, are metered in, the acidic catalyst required for the reaction, preferably para-toluene sulfonic acid, being added before trimethylolpropane and acrylic acid are metered in.

The reaction temperature is brought to not more than 120°, preferably not more than 110° C., particularly preferably not more than 100° C., and the duration of the reaction is less than 20, preferably less than 15, particularly preferably less than 10, hours.

The reaction mixture is then, if required, prewashed, neutralized and, if required, subsequently washed and the solvent is then removed by distillation and stripping to a content of less than 0.5, preferably less than 0.3, % by weight.

The reaction mixture thus obtained and containing substantially trimethylolpropane triacrylate has a color number of 150 APHA or less, preferably 100 APHA or less, particularly preferably 80 APHA or less, and a viscosity (according to DIN 51562 at 25° C.) of less than 160, preferably less than 140, particularly preferably less than 120, mPa.s.

The reaction mixture may contain, for example, trimethylolpropane monoacrylate, trimethylolpropane diacrylate, trimethylolpropane and oxyesters of trimethylolpropane and the acrylates as further components in addition to trimethylolpropane triacrylate.

Of course, the polyhydric alcohols mentioned in this document and containing less than 500 ppm of bound formaldehyde also have advantages when used in the esterification with acids other than (meth)acrylic acid, for example in the esterification with organic carboxylic acids, preferably in the esterification with long-chain, saturated or unsaturated fatty acids and particularly preferably α,β-unsaturated carboxylic acids, such as (meth)acrylic acid, crotonic acid, maleic acid or fumaric acid.

Unless stated otherwise, ppm and percentage data used in this document are based on weight.

APHA color numbers were determined according to DIN-ISO 6271.

Unless stated otherwise, viscosities were determined according to DIN 51562 at 25° C.

### EXAMPLES

The column DB5 (from J&W Scientific) having a length of 30 m, a diameter of 0.32 mm and 1 μm coating thickness was used for the gas chromatographic determination of the contents of bound formaldehyde stated in this application. Detection was effected using a flame ionization detector.

### Example 1

729 g of acrylic acid, 1 907 g of trimethylolpropane (content of bound formaldehyde, based on IVa, IVb (n=1), IVb (n=2) and IVc where R¹=ethyl and R²=hydroxymethyl: 282 ppm), 56.9 g of aqueous stabilizer solution (containing 1.1 g of para-methoxyphenol and 1.5 g of hypophosphorous acid), 18.3 g of 40% strength aqueous CuCl₂ solution, 25.6 g of 96% strength sulfuric acid and 2 514 g of cyclohexane were initially taken in a 10 l reactor having an external natural circulation evaporator, distillation column, condenser and phase separator. The natural circulation evaporator was a tube-bundle heat exchanger heated by means of heat transfer oil. The tube-bundle heat exchanger consisted of three tubes, and each tube had a length of 700 mm and a diameter of 9 mm. The forward-flow temperature of the heat transfer oil was 150° C. and the oil circulation was regulated manually. In addition, air was passed into the natural circulation evaporator, the amount of air being 40 l/h. After the water initially taken with the starting material had evaporated, the amount of air was reduced to 20 l/h and 2 441 g of acrylic acid was subsequently metered. The amount of air was then reduced to 2 l/h. The cyclohexane reflux was metered to the distillation column and regulated by means of the internal temperature of the reactor. The minimum reflux was 1 600 g/h. The reaction temperature was increased to 95° C. in the course of 40 minutes. After an esterification time of 510 minutes, the experiment was terminated. Altogether, 735 g of aqueous phase were discharged and 5 049 g of crude ester were obtained. The aqueous phase discharged contained 5.6% of acrylic acid. The crude ester contained 6.0% of acrylic acid.

Thereafter, 1 620 g of cyclohexane were added to the crude ester and the mixture was washed three times as follows:
1. 1 100 g of 7% strength sodium chloride solution
2. 1 550 g of 13% strength sodium hydroxide solution
3. 1 360 g of 26% strength sodium chloride solution

Thereafter, 0.9 g of para-methoxyphenol was added, cyclohexane was taken off at 60° C./10 mbar and filtration was effected (Seitz filter K3).

The end product had a density of 1.1041 g/cm³ (23° C.) and a dynamic viscosity of 85 mPa.s according to DIN 51562 at 23° C.

### Comparative Example 1

Example 1 was repeated. Trimethylolpropane having a content of bound formaldehyde, based on IVa, IVb (n=1), IVb (n=2) and IVc where R¹=ethyl and R²=hydroxymethyl, of 1 400 ppm was used.

Altogether, 782 g of aqueous phase were discharged and 5 158 g of crude ester were obtained. The aqueous phase discharged contained 6.7% of acrylic acid. The crude ester contained 4.0% of acrylic acid.

The end product had a density of 1.1153 g/cm³ (23° C.) and a dynamic viscosity of 246 mPa.s according to DIN 51562 at 23° C.

1. A process for the preparation of (meth)acrylates of polyhydric alcohols by reacting (meth)acrylic acid and the corresponding polyhydric alcohol in the presence of at least one acidic catalyst and, if required, at least one polymerization inhibitor and in the presence or absence of a solvent, wherein the polyhydric alcohol used contains less than 500 ppm of bound formaldehyde.

2. A process as claimed in claim 1, wherein the polyhydric alcohol is an alcohol of the formula (I), 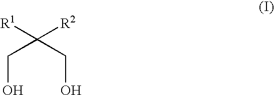
where R¹ and R², independently of one another, are hydrogen, C₁-C₁₀-alkyl, C₁-C₁₀-hydroxyalkyl, carboxyl or C₁-C₄-alkoxycarbonyl.

3. A process as claimed in claim 1, wherein the alcohol is an alkoxylated alcohol which prepared by
reacting a polyhydric alcohol containing less than 500 ppm of bound formaldehyde with at least one alkylene oxide.

4. A process as claimed in claim 1, wherein the polyhydric alcohol is trimethylolbutane, trimethylolpropane, trimethylolethane, neopentylglycol, pentaerythritol, 2-ethyl-1,3-propanediol, 2-methyl-1,3-propanediol, 1,3-propanediol, dimethylolpropionic acid, methyl dimethylolpropionate, ethyl dimethylolpropionate, dimethylolbutyric acid, methyl dimethylolbutyrate or ethyl dimethylolbutyrate.

5. A process as claimed in claim 2, wherein the polyhydric alcohol has been obtained by a process, which comprises:
reacting an aldehyde of the formula (II), 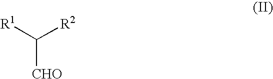
where R¹ and R² have the meanings as stated above, with formaldehyde and then
converting the aldehyde group into a hydroxyl group by catalytic hydrogenation.

6. A process as claimed in claim 1, which further comprises:
purifying the polyhydric alcohol by distillation, then
heating the purified polyhydric alchol, and then
purifying the polyhydric alcohol.

7. A process as claimed in claim 6, wherein the heating occurs from 100 to 300° C.

8. A process as claimed in claim 1, wherein, in the esterification, the molar ratio of the number of hydroxyl groups in the polyhydric alcohol to (meth)acrylic acid is 1:1.0-2.0.

9. A process as claimed in claim 1, wherein the acidic catalyst used in the esterification is sulfuric acid, methanesulfonic acid and/or p-toluenesulfonic acid or a mixture thereof.

10. A process as claimed in claim 1, wherein the polymerization inhibitor (mixture) used comprises at least one compound selected from the group consisting of phenothiazine, hydroquinone, hydroquinone monomethyl ether, 2-tert-butyl-4-methylphenol, 6-tert-butyl-2,4-diemthylphenol, 2,6-di-tert-butyl-4-methyphenol, 2-tert-butylphenol, 4-tert-butylphenol, 2,4-di-tert-butylphenol, 2-methyl-4-tert-butylphenol, 4-tert.-butyl-2,6-dimethylphenol, hypophosphorous acid, copper acetate, copper chloride, copper salicylate, and mixtures thereof.

11. (Cancelled).

12. A method, which comprises:
esterifying an acid with a polyhydric alcohol comprising less than 500 ppm of bound formaldehyde.

13. An ester, prepared by the process as claimed in claim 12.
